(19) United States
(12) Patent Application Publication
(10) Pub. No.: US 2024/0139216 A1
(43) Pub. Date: May 2, 2024

(54) STABLE ORAL CANNABIDIOL COMPOSITIONS

(71) Applicant: CARDIOL THERAPEUTICS INC., Oakville (CA)

(72) Inventors: Blagoja RISTEVSKI, Oakville, Ontario (CA); Anthony Ernest BOLTON, Oakville, Ontario (CA)

(73) Assignee: CARDIOL THERAPEUTICS INC., Oakville, Ontario (CA)

(21) Appl. No.: 18/265,509

(22) PCT Filed: Dec. 7, 2020
(86) PCT No.: PCT/CA2020/051680

§ 371 Date: Jun. 6, 2023

(51) Int. Cl. A61K 31/00; A61K 47/14; A61K 47/22; A61K 47/44

(52) U.S. Cl. CPC A61K 31/658; A61K 47/14; A61K 47/22; A61K 47/44

(57) ABSTRACT

Stabilized oral cannabidiol composition comprising cannabidiol (CBD) having a purity of at least 98%. The composition further contains β-caryophyllene (BCP), at least one additional lipophilic solvent (e.g. medium chain triglycerides (MCT)), and at least one additional antioxidant (e.g. α-tocopherol (vitamin E)). The composition is substantially free of THC. The CBD can be synthetic, biosynthetic or of botanical origin. Also disclosed are methods of making and the stabilized oral CBD composition.

### FIELD

The present specification relates to oral cannabinoid compositions.

### BACKGROUND

Many studies have demonstrated the therapeutic potential of cannabinoid therapies. As a result, cannabinoids have been used or explored for use in treating various diseases, conditions, disorders and/or their symptoms, including chronic pain, neuropathic pain, epilepsy and the like (e.g. Dravet Syndrome, Lennox Gastaut Syndrome, myoclonic seizures, juvenile myoclonic epilepsy, refractory epilepsy, juvenile spasms, West syndrome, refractory infantile spasms, infantile spasms), tuberous sclerosis complex, brain tumors (e.g. Glioblastoma multiforme, or GBM), cannabis use disorder, post-traumatic stress disorder, anxiety, early psychosis, schizophrenia, Alzheimer's Disease, autism, and withdrawal from opioids, cocaine, heroin, amphetamines, and nicotine.

There are at least 113 different cannabinoids that occur naturally in, and can be isolated from, the Cannabis sativa plant, exhibiting varied effects. These include tetrahydrocannabinol (THC), cannabidiol (CBD) and cannabinol (CBN). THC is the principal psychoactive constituent, whereas cannabidiol (CBD) is non-intoxicating making it highly desirable for use in therapeutic compositions, especially those intended for medical applications.

CBD can be administered in a variety of ways including orally. To be useful and approved as a pharmaceutical product, compositions employing CBD as the active ingredient must provide safe and consistent dosing and be therapeutically effective. However, a challenge with oral based therapies is instability of the CBD in the composition. For example, CBD can degrade during storage and be converted to other products such as cannabinol (CBN), THC, quinone, and CBDO, etc. This can obviously result in variable potency, imprecise dosing, and an unpredictable response.

Thus, a need exists to stabilize CBD in a composition intended for oral administration. A need also exists to keep the level of THC (present as an impurity) to as low a level as possible to not only meet government regulations that limit the amount of THC but also to reduce the risk of psychotropic effects. The present invention is intended to meet these needs.

### SUMMARY OF INVENTION

Surprisingly, the inventors have found that β-caryophyllene (BCP) is effective to both solubilize and stabilize CBD in a composition according to the present invention such that an acceptable shelf-life can be obtained with very few other ingredients. Both CBD and BCP have been demonstrated to have anti-inflammatory properties. Therefore, using both in the same formulation is also expected to provide an enhanced anti-inflammatory effect.

Thus, the present invention provides, according to a first aspect, a stable oral cannabidiol composition comprising, consisting essentially of, or consisting of (a) cannabidiol (CBD); (b) a lipophilic carrier consisting of β-caryophyllene (BCP) and at least one additional lipophilic solvent for CBD (e.g. medium chain triglycerides (MCT)); (c) at least one additional antioxidant (e.g. α-tocopherol (vitamin E)); and (optionally) an effective amount of at least one pharmaceutically acceptable excipient, wherein the composition is substantially free of THC.

The CBD can be synthetic or of natural origin, i.e. chemically synthesized, biosynthetic, or sourced botanically and rigorously purified. Regardless of the type of CBD used, the CBD must have a purity of at least 98, 98.5, 99, 99.5, 99.6, 99.7, 99.8, or 99.9 wt. %.

In some embodiments, the composition is substantially free of ingredients or compounds (other than those described as being present) that are cannabinoids, terpenes or essential oils, solvents, absorption enhancers, surfactants, emulsifiers, water, alcohols (including ethanol and glycols such as polyethylene glycols), delivery agents, stabilizers, gelling agents (e.g. alginate/algin/alginic acid, and agar), pharmaceutically active ingredients, and vitamins. For example, the present compositions can be substantially free of at least 1, 2, 3, 4, 5, 6, 7, 8, 9, 10, 11, 12, 13, or all of the above. For example, the present composition can exclude bergamotenes (e.g. α-trans-bergamotene, α-cis-bergamotene), bisabolenes (e.g. trans-γ-bisabolene, cis-γ-bisabolene), bisabolols (e.g. α-bisabolol, epi-α-bisabolol), borneol, α-cadinene, camphene, carenes (e.g. delta-3-carene), α-caryophyllene, caryophyllene oxide, citronellol, γ-curcumene, p-cymene, β-elemene, eucalyptol, eudesmols (e.g. α-eudesmol, β-eudesmol, γ-eudesmol), famesenes (e.g. α-farnesene, cis-β-farnesene), fenchols (e.g. β-fenchol), fenchone, geraniol, guaiol, gualenes (e.g. α-gualene), humulenes (e.g. α-humulene), ipsdienol, isophytol, isopulegol, linalool, limonene, α-longipinene, menthol, γ-muurolene, myrcene, nerolidol, ocimenes (e.g. trans- and cis-ocimenes), phellandrenes (e.g. α-phellandrene, β-phellandrene), phytol, pinenes (e.g. α-pinene, β-pinene), pulegone, sabinenes (e.g. cis-sabinene hydrate), selinenes (e.g. α-selinene, β-selinene), γ-terpinene, terpinene, terpineol, terpinolene, α-thujene, valencene, α-ylangene, cannabidolic acid (CBDA), cannabigerolic acid (CBGA), cannabinol (CBN), cannabinolic acid (CBNA), cannabigerol (CBG), cannabichromene (CBC), cannabichromenic acid (CBCA), cannabicyclol (CBL), cannabicyclolic acid (CBLA), cannabivarin (CBV), tetrahydrocannabivarin (THCV), cannabidivarin (CBDV), cannabichromevarin (CBCV), cannabigerovarin (CBGV), tetrahydrocannabinolic acid (THCA), cannabigerol monomethyl ether (CBGM), cannabielsoin (CBE), cannabicitran (CBT), cannabidiol-dimethylheptyl (CBD-DMH), and N-[8-(2-hydroxybenzoyl) amino] caprylate (SNAC).

Pharmaceutically active agents that can be expressly excluded from the present compositions include opioid receptor agonists (e.g. opioid analgesics).

In some embodiments, the present composition exclude the following absorption enhancers—Gelucire™ 44/14; Gelucire™ 50/13; Tagat™ TO; Tween™ 80; isopropyl myristate, polysorbates, sorbitan esters, poloxamer block copolymers, PEG-35 castor oil, PEG-40 hydrogenated castor oil, caprylocaproyl macrogol-8 glycerides, PEG-8 caprylic/capric glycerides, sodium lauryl sulfate, dioctyl sulfosuccinate, polyethylene lauryl ether, ethoxydiglycol, propylene glycol mono-di-caprylate, glycerol monocaprylate, glyceryl fatty acids (C8-C18) ethoxylated, oleic acid, linoleic acid, glyceryl caprylate/caprate, glyceryl monooleate, glyceryl monolaurate, caprylic/capric triglycerides, ethoxylated nonylphenols, PEG-(8-50) stearates, olive oil PEG-6 esters, triolein PEG-6 esters, lecithin, d-α tocopheryl polyethylene glycol 1000 succinate, polycarbonate, sodium glycocholate, sodium taurocholate, cyclodextrins, citric acid, sodium citrate, triacetin, combinations thereof, and the like. However, if used, the absorption enhancer may be present in an amount of from about 0.001% w/v to about 10% w/v, or from about 0.01% w/v to about 5% w/v.

In the same or other embodiments, the CBD is present in an amount from about 1% w/v to about 35% w/v; the lipophilic carrier is present in an amount from about 60% w/v to about 98.9% w/v; the volume ratio of BCP to the at least one additional lipophilic solvent is from about 1:1 to about 1:5; and the at least one antioxidant is present in an amount from about 0.1% w/v to about 5% w/v.

In some embodiments, the CBD can be present in an amount from about 1, 2, 3, 4, 5, 6, 7, 8, or 9% w/v and up to about 35, 34, 33, 32, 31, 30, 29, 28, 27, 26, 25, 24, 23, 22, 21, 20, 19, 18, 17, 16, 15, 14, 13, or 12% w/v. In the same or other embodiments, the compositions can contain CBD in an amount from about 10, 20, 30, 40, 50, 60, 70, 80, 90, or 100 mg CBD/mL of the composition and up to about 350, 325, 300, 275, 250, 225, 200, 175, and 150 mg CBD/mL of the composition. For example, the CBD can be present in an amount of 25, 50, 75 or 100 mg CBD/mL of the composition.

The volume ratio of the BCP to the at least one additional lipophilic solvent can vary to all values within the above range, such as from about 1:1 to about 1:4; from about 1:1 to about 1:3, and/or about 1:2.

The at least one additional lipophilic solvent can be selected from the group consisting of medium chain (C6-C12) triglycerides (MCT), coconut oil, sesame oil, fish oil, avocado oil, oils from nuts and seeds, corn oil, peanut oil, safflower oil, soybean oil, and palm kernel oil. In some embodiments, the at least one additional lipophilic solvent consists of a mixture of C8 and C10 triglycerides. The weight ratio of the C8 triglyceride to the C10 triglyceride can be from about 35:65 to about 85:15, about 45:55 to about 75:25, or from about 55:45 to about 65:35. In other embodiments, the at least one additional lipophilic solvent can consist of coconut oil.

The at least one additional antioxidant can be selected from the group consisting of α-tocopherol (vitamin E), polyphenols, carotenoids, propyl gallate, lecithin, curcumin, sesamin, sesamol, sesamolin, ascorbyl palmitate, butylated hydroxyanisole (BHA), butylated hydroxytoluene (BHT), and monothioglycerol tert-butylhydroquinone (TBHQ). In some embodiments, α-tocopherol (vitamin E) is present in an amount from about 0.3, 0.5, or 0.7 and up to about 1.5, 1.3, or 1.1% w/v. The α-tocopherol can also be present in an amount from about 0.75% w/v to about 1.25% w/v.

In a particular embodiment, the composition consists of:
a. CBD having a purity of at least 99.5 wt. %, in an amount from about 2% w/v to about 12% w/v;
b. BCP in an amount from about 28% w/v to about 30% w/v;
c. a mixture of C8 and C10 triglycerides in an amount from about 58.5% w/v to about 67.5% w/v, wherein the weight ratio of the C8 triglyceride to the C10 triglyceride is from about 55:45 to about 65:35;
d. α-tocopherol (Vitamin E) in an amount from about 0.5% w/v to about 1.5% w/v; and
e. (optionally) from 0% w/v to an effective amount of at least one pharmaceutically acceptable excipient;
wherein THC is absent or is present as an impurity in an amount less than 10 ppm based on the total composition.

In some embodiments, the CBD is a botanical CBD isolate with less than 2, 1.5, 1, 0.7 or 0.5 wt. % total impurities based on the weight of the CBD. In the same or other embodiments, THC can be as low as 0.009 mg per gram CBD. A botanical CBD isolate having this low level of THC is available from Dalton Pharma Services of Toronto, Ontario, Canada (“Dalton Pharma CBD”). The Dalton Pharma CBD also contains less than 0.3 wt. % of other cannabinoids, including cannabidivarin (CBDV) and cannabidibutol (CBDB). Such CBD is considered to be purer than other botanical CBD isolates on the market which contain much higher levels of these impurities. CBDV and CBDB are known to be structurally similar to CBD and considered more difficult to separate from CBD during purincation.

In other embodiments, the CBD is made synthetically, as will be discussed further below.

According to a second aspect, the invention provides a method of stabilizing cannabidiol (CBD) in an oral composition according to the first aspect, the method comprising mixing an effective amount of CBD having a purity of at least 98, 98.5, 99, 99.5, 99.6, 99.7, 99.8, or 99.9 wt. % with BCP and at least one additional lipophilic solvent until the CBD is dissolved in the solvents, then adding at least one additional antioxidant and (optionally) at least one pharmaceutically acceptable excipient, wherein the amounts of the compounds or ingredients that are used to make the composition are selected to provide a composition according to the first aspect.

These and other aspects of the invention will be described more fully below.

### DETAILED DESCRIPTION

### Definitions

For the sake of clarity and to avoid ambiguity, certain terms are defined herein as follows.

The term “pharmaceutically active agent” means any composition of matter (e.g. agent, compound, or ingredient) capable of providing a therapeutic effect (e.g. healing, alleviating, preventing and/or slowing the progression of a disease, condition, or their symptoms) to a subject, including drugs, cells, DNA, RNA, oligonucleotides, proteins, and peptides.

The present specification describes the purity of CBD in terms of “X” wt. %. This means that the CBD may contain up to 100−X % by weight of impurities (compounds other than CBD), based on the weight of the CBD. When the purity of other ingredients is described, similar principles will apply. The purity of an ingredient, e.g. CBD, can be determined by high performance liquid chromatography (HPLC), e.g. by the area normalization of an HPLC or GC-FID profile. Thus, reference to CBD having a purity of at least 99.7 wt. % means that the CBD may contain up to 0.3 wt. % impurities based on the weight of the CBD. Since the present composition contains compounds/ingredients other than CBD, the impurities present in the CBD will be present in a much smaller amount in terms of wt. %, based on the weight of the composition.

The term “subject” means members of the animal kingdom including humans and other mammals.

When used herein, the term “treatment” and “prevention” are intended to include “improving quality of life” or “extending the life” of a subject and not necessarily “curing” a condition, disorder or disease.

“Pharmaceutically acceptable excipient” when used herein means any substance which can be formulated with or that is present alongside a pharmaceutically active agent to achieve a desired function or functions. Examples include colouring agents, flavouring agents, and preservatives. To be “pharmaceutically acceptable”, the excipient must be non-toxic, safe for human and/or animal consumption, and compatible with the other ingredients in the composition, having regard to the oral mode of administration. The person skilled in the art will appreciate what compounds or ingredients would qualify as a pharmaceutically acceptable excipient given the teachings of the present specification and information in the public domain.

As used herein, the terms “stability,” “stable” or the like, when used in association with compositions according to the present invention, mean that there is no statistically significant loss of CBD in the composition when stored in a sealed container, away from light, at 40° C.±2° C. and at relative humidity (RH) 75%+5% for at least eight (8) weeks. When stored in a refrigerator at a temperature of from about 5° C.±3° C., such compositions can be expected to be stable for a longer period, assuming all other conditions are the same. Compositions according to the present invention remain as a clear uniform liquid that will allow consistency of dosing.

The phrases “at least one,” “one or more,” and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The terms “a” or “an” entity refers to one or more of that entity. As such, the terms “a” (or “an”), “one or more” and “at least one” can be used interchangeably herein. It should also be noted that the term “or” is generally employed in the sense of “and/or” unless the context clearly dictates otherwise.

The term “comprising” means “including without limitation.” Thus, a composition comprising a list of ingredients may include additional ingredients not expressly recited. It is also to be noted that the terms “comprising,” “including,” and “having” can be used interchangeably.

The term “consisting of” means “including the listed ingredients and such additional ingredients as may be present in the listed ingredients as natural or commercial impurities or additives.” Natural and commercial impurities and additives will be apparent to the person of ordinary skill in the art.

The term “consisting essentially of” means “including the listed ingredients (including natural or commercial impurities and typical commercial additives to said ingredients) and any additional ingredients that do not materially affect the basic and novel properties.” By “basic and novel' properties” is meant the stability of the CBD in the composition and the presence of THC in an amount less than 0.3 wt. % based on the total composition.

Unless stated otherwise, the terms “wt. %,” “% w/w,” and variations thereof, are used interchangeably herein and refer to the amount of a substance as the weight of that substance divided by the total weight of the composition that contains the substance, and multiplied by 100.

The term “% w/v” means “percent weight/volume” and is calculated according to the following formula using the gram as the base measure of weight (w):

% w/v=g of ingredient or compound/100 mL of solution

Unless otherwise specified, a composition that is “substantially free” of “Y” means that “Y” is either not present or is present in an amount less than 0.3 wt. % based on the composition.

The term “about” refers to variations in an expressed numerical quantity that can occur, for example, through measuring and liquid handling procedures used for making pharmaceutical compositions, differences in the manufacture, source, or purity of the ingredients used to make the compositions, and/or differences due to different equilibrium conditions or different reaction levels for a composition resulting from an initial mixture. For the sake of clarity, the term “about” includes variations in the expressed value up to ±5%. Whether or not a value is modified by the term “about,” the claims include equivalents to the values.

When used herein, the term “effective amount,” when used to specify an amount of an ingredient, means that amount which would bring about the desired effect given the purpose and function of the ingredient in a composition and also the purpose and function of the composition containing the ingredient. What constitutes an effective amount will be determinable by the person of ordinary skill in the art without having to engage in inventive experimentation.

The values recited herein are intended to include all values that meet the stated parameters including those not expressly recited. Thus, for example, a value of less than 1.0 is intended to include less than 0.99, less than 0.98, less than 0.97, less than 0.90, less than 0.84, less than 0.56, less than 0.01, etc. Thus, all ranges disclosed herein are to be understood to encompass any and all subranges subsumed therein. For example, a stated range of “1 to 10” should be considered to include any and all subranges between (and inclusive of) the minimum value of 1 and the maximum value of 10, e.g., 1 to 6.3, or 5.5 to 10, or 2.7 to 6.1, etc.

The present specification contemplates the possibility of omitting any components listed herein. The present specification further contemplates the omission of any components even though they are not expressly named as included or excluded from the specification.

The chemical structures herein are drawn according to the conventional standards known in the art. Thus, where an atom, such as a carbon atom, as drawn appears to have an unsatisfied valency, then that valency is assumed to be satisfied by a hydrogen atom, even though that hydrogen atom is not necessarily explicitly drawn. The structures of some of the compounds of this specification include stereogenic carbon atoms. It is to be understood that isomers arising from such asymmetry (e.g., all enantiomers and diastereomers) are included within the scope of this specification unless indicated otherwise. That is, unless otherwise stipulated, any chiral carbon center may be of either (R)- or (S)-stereochemistry. Such isomers can be obtained in substantially pure form by classical separation techniques and by stereochemically-controlled synthesis. Furthermore, alkenes can include either the E- or Z-geometry, where appropriate.

Oral Cannabidiol Composition

The present compositions contain cannabidiol (CBD) as the active ingredient. CBD is the compound shown below:

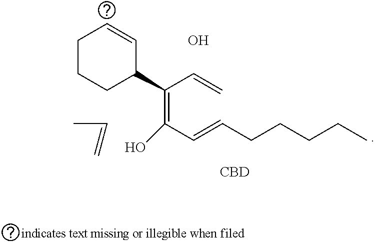

The CBD used in the present compositions are synthetic or of natural origin. Synthetic CBD is manufactured using chemical means. Methods of manufacturing synthetic cannabidiol are known in the art. For example, the CBD from Noramco, Inc. headquartered in Wilmington Delaware, U.S.A. (now Purisys Inc. headquartered in Athens, Georgia) is made according to processes such as those described in U.S. patent publication US 2017/0008868 A1 (granted as U.S. Pat. No. 10,059,683) and US 2018/031,976 A1. These references are incorporated herein by reference. Synthetic CBD made by other processes and manufacturers can also be used in the present compositions.

Biosynthetic CBD is CBD that is derived from genetically modified yeasts, bacteria, and the like and grown in culture. With biosynthesis, each genetically altered cell acts as a factory to produce large amounts of CBD, making this method potentially more efficient, less costly, and scalable.

Botanically sourced CBD can be derived from a variety of plants including marijuana and hemp using known extraction and purification methods.

Regardless of the type of CBD that is used to make the present composition, the CBD used herein has a purity of at least 98, 98.5, 99, 99.5, 99.6, 99.7, 99.8, or 99.9 wt. %. Furthermore, THC will not be present or, if present, will not exceed 0.3 wt. %, and preferably will not exceed 0.2 wt. %, based on the weight of the CBD. In some embodiments, the THC will not be detectable in the CBD.

The skilled person will appreciate that commercial sources of synthetic CBD will contain minor amounts of impurities such as residual solvents and by-products of manufacture, e.g. olivetol, monobromo-CBD, and delta-9-THC. Residual solvents include methanol, n-heptane, dichloromethane, and triethylamine.

Impurities that may be present in CBD (including synthetic and botanical CBD) include THC as well as other cannabinoids. The chemical structure of THC is shown below:

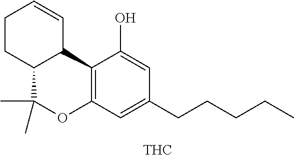

THC is also sometimes referred to as delta-9-tetrahydrocannabinol and delta-9-THC. When used herein, THC is intended to include its double bond isomers and their stereoisomers.

In some embodiments, the CBD used in the present compositions contains an “ultra-low” level of THC. This means that THC is not present or not detectable, or, if it is detectable, it is present in an amount not exceeding 0.0009 wt. % based on the weight of the CBD.

In the same or other embodiments, the level of each of THC in an oral CBD composition according to the invention will not exceed 100, 90, 80, 70, 60, 50, 40, 30, 20, or 10 ppm based on the total composition. Preferably, the level of THC is reduced to as low as possible. Even more preferably, the amount of THC is less than 10 ppm based on the total (final) composition.

In still the same or other embodiments, the amount of CBDB and CBDV in the purified CBD will each not exceed 0.3 wt. % based on the CBD. In yet other embodiments, the total amount of CBDB and CBDV will not exceed 0.3 wt. % based on the CBD.

Solvents for CBD

Compositions according to the invention will contain at least two solvents in an amount effective to solubilize the CBD in the composition. The solvent must be lipophilic and generally recognized as safe (GRAS) and suitable for human or animal consumption. One of the solvents is β-caryophyllene (BCP), also named trans-(1R,9S)-8-Methylene-4,11,11-trimethylbicyclo[7.2.0]undec-4-ene or [1R-(1R,4E,9S)]-4,11,11-trimethyl-8-methylene-bicyclo[7.2.0]undec-4-ene, is a natural bicyclic sesquiterpene compound found in botanical extracts of plants, including Cannabis sativa. It is a constituent of many essential oils, especially clove Syzygium aromaticum oil and is “generally recognized as safe” (GRAS).

Caryophyllene is the only terpene known to interact with the endocannabinoid system (at CB2 receptors). β-caryophyllene selectively binds to the CB2 receptor and is a functional CB2 agonist. Furthermore, β-caryophyllene has been identified as a functional non-psychoactive CB2 receptor ligand in foodstuff and as a macrocyclic anti-inflammatory cannabinoid in cannabis. (Proc Natl Acad Sci USA. 2008 Jul. 1; 105(26):9099-104. doi: 10.1073/pnas.0803601105. Epub 2008 Jun. 23. β-caryophyllene is a dietary cannabinoid). BCP can be used as a flavoring agent, antioxidant, penetration enhancer via gastrointestinal mucosa, anti-inflammatory agent, and solvent (see, e.g., WO2002034294 to Schwankl et al). It may be useful in improving the systemic availability of CBD (WO2002034275A1 to Schwankl et al.). It is predicted that compositions according to the present invention will provide enhanced absorption of CBD into the subject's bloodstream and therefore enhanced therapeutic efficacy. This is based on the theory that BCP can block or inhibit enzymes released by bacteria in the gut that expedite degradation of CBD. By impeding CBD degradation in the gastrointestinal tract, a greater percentage of the administered dose of CBD should become bioavailable.

Surprisingly, the inventors have found that BCP is a highly effective solvent and stabilizer for CBD when used in combination with at least one additional lipophilic solvent in the amounts described herein. For example, at least 300 mg of CBD can be dissolved in 1 mL of BCP. The enhanced stability of the CBD provides a composition with an acceptable shelf life and which can provide consistency in dosing and enhanced safety.

Without being bound by theory, it is believed that the presence of BCP in the amounts disclosed herein impedes degradation of the at least one additional lipophilic compound (e.g. MCT) into free fatty acids which can interact with moisture (either present as an impurity in the ingredients used to make the composition or in the atmosphere) to produce an acidic environment that can cause CBD to convert to THC. The BCP in the present compositions appears to act synergistically with vitamin E to impede this conversion thereby providing a more stable CBD product that has ultra-low levels of THC.

When used herein, “BCP” means commercial sources of BCP that are “food grade” and generally recognized as safe (“GRAS”). Such products are typically made by purifying plant extracts and can therefore contain a multiplicity of compounds. For example, embodiments of the invention described below use BCP from Sigma Aldrich. The product specification for this product is included in ANNEX A (CAS Number 87-44-5). As can be seen from the product specification, the Sigma Aldrich BCP contains at least 95% w/w major and minor C15H24 terpenes and up to 5% w/w impurities such as by-products of manufacturing. Thus, the BCP from Sigma Aldrich has a “purity” of at least 95%. BCP from Vigon International, Inc. (of Stroudsburg, Pennsylvania, U.S.A.) has a purity ranging from 90-100% w/w (Vigon Code 500796; CAS #87-44-5). The present invention contemplates using BCP having a purity of at least 90% w/w, e.g. at least 90.5, 91, 91.5, 92, 92.5, 93, 93.5, 94, 94.5, 95, 95.5, 96, 96.5, 97, 97.5, 98, 98.5, and 99% w/w.

The relative amounts of BCP to CBD can vary. For example, for every 1 mL of BCP, the amount of the CBD can vary from about 1 mg, 2 mg, 5 mg, or 10 mg, and up to about 300 mg, 275 mg, 200 mg, or 100 mg. The weight ratio of CBD to BCP can be about 1:5, 1:4, 1:3, 1:2, or 1:1.

The present compositions also contain BCP together with at least one additional lipophilic solvent. The volume ratio of BCP to the at least one additional lipophilic solvent can be anywhere from about 1:1 to about 1:5, as noted above, including about 1:2. The at least one other solvent can be at least one oil selected from the group consisting of avocado oil, canola oil, coconut oil, corn oil, fish oil, hemp oil, medium chain (C6-C12) triglycerides (MCTs) (both natural and synthetic), blends of MCTs and long chain triglycerides (LCTs), olive oil, palm oil, palm kernel oil, peanut oil, safflower oil, soybean oil, structured lipids, almond oil, beech nut oil, brazil nut oil, cashew oil, cottonseed oil, grapeseed oil, hazelnut oil, macadamia oil, mongongo nut oil, pecan oil, pine nut oil, pistachio oil, pumpkin seed oil, sesame oil, rapeseed oil, sunflower oil, and walnut oil.

One or more other edible oils can also be used as the at least one solvent. These include amaranth oil, apricot oil, apple seed oil, argan oil, babassu oil, ben oil, Borneo tallow nut oil, cape chestnut oil (also called yangu oil), carob pod oil, cocoa butter, cocklebur oil, cohune oil, coriander seed oil, date seed oil, dika oil, false flax oil, kapok seed oil, kenaf seed oil, lallemantia oil, mafura oil, marula oil, meadowfoam seed oil, mustard oil, Niger seed oil, poppyseed oil, nutmeg butter, okra seed oil, papaya seed oil, perilla seed oil, persimmon seed oil, pequi oil, pili nut oil, pomegranate seed oil, poppyseed oil, pracaxi oil, prune kernel oil, quinoa oil, ramtil oil, rice bran oil, royle oil, shea butter, sacha inchi oil, sapote oil, seje oil, taramira oil, tea seed oil (Camellia oil), thistle oil, tigernut oil (or nut-sedge oil), tobacco seed oil, tomato seed oil, wheat germ oil, and oils from the seeds of melons and gourds (e.g. bitter gourd oil, bottle gourd oil, buffalo gourd oil, butternut squash seed oil, egusi seed oil, pumpkin seed oil, and watermelon seed oil).

The skilled person will recognize that many of the oils listed above are oils from nuts and seeds.

Coconut oil contains not only MCT (medium chain triglycerides) predominantly, but also contains LCT (long chain triglycerides) in an amount of about 10% w/w. The presence of LCT promotes secretion of the hormone cholecystokinin (CCK) which promotes the formation of chylomicrons which facilitate the lymphatic uptake of lipophilic molecules. This can help to avoid the first pass effect of degradation of CBD by the liver.

In one embodiment, the solvent comprises one or more medium chain triglycerides (MCTs). The medium chain triglyceride may be synthetic or natural (e.g., produced from fractionated oils, such as coconut oil and/or palm kernel oil). “Medium chain triglyceride” refers to esters of glycerol having three C6 to C12 fatty acid chains, where the three fatty acid chains may be the same or different. Medium chain triglycerides are represented by the following formula:

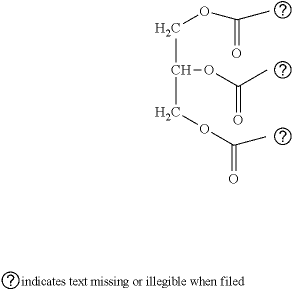

wherein each x is independently 4, 6, 8, or 10. When x is 4, the chain is referred to as a C6 fatty acid. When x is 6, the chain is referred to as a C8 fatty acid. When x is 8, the chain is referred to as a C10 fatty acid. When x is 10, the chain is referred to as a C12 fatty acid. In various embodiments, each x is the same integer; two x are the same integer and one x is a different integer; or each x is a different integer.

In various embodiments, the medium chain triglyceride comprises esters of (i) three C8 fatty acids; (ii) three C10 fatty acids; (iii) two C8 fatty acids and one C10 fatty acid; (iv) two C10 fatty acids and one C8 fatty acid; (v) two C8 fatty acids and one C6 fatty acid; (vi) two C10 fatty acids and one C6 fatty acid; (vii) one C8 fatty acid, one C10 fatty acid, and one C6 fatty acid; or (viii) any other combination of C6, C8, C10, and C12 fatty acids.

The skilled artisan will appreciate that a mixture of medium chain triglycerides may result from any process (e.g., fractionation, hydrogenation) used to prepare medium chain triglycerides. For example, substantially all the medium chain triglycerides obtained from fractionated coconut oil may comprise C8 and/or C10 fatty acids; however, there may be some medium chain triglycerides containing C6 and/or C12 fatty acids.

In one embodiment, the medium chain triglycerides comprise esters of (i) 0 to 2% w/w C6 fatty acid, 65 to 80% w/w C8 fatty acid, 20 to 35% w/w C10 fatty acid, and 0 to 2% w/w C12 fatty acid; (ii) 0 to 2% w/w C6 fatty acid, 50 to 65% w/w C8 fatty acid, 30 to 45% w/w C10 fatty acid, and 0 to 2% w/w C12 fatty acid; (iii) 0 to 2% w/w C6 fatty acid, 45 to 65% w/w C8 fatty acid, 30 to 45% w/w C10 fatty acid, 0 to 3% w/w C12 fatty acid, and 0 to 5% w/w linoleic acid; or (iv) 0 to 2% w/w C6 fatty acid, 45 to 55% w/w C8 fatty acid, 30 to 40% w/w C10 fatty acid, 0 to 3% w/w C12 fatty acid, and 10 to 20% w/w succinic acid. In one embodiment, the medium chain triglyceride comprises 0 to 2% w/w C6 fatty acid, 50 to 65% w/w C8 fatty acid, 30 to 45% w/w C10 fatty acid, and 0 to 2% w/w C12 fatty acid, and which is commercially available as MIGLYOL® 812 (Sasol Germany GmbH, Witten, Germany). The weight % values recited in this paragraph are based on the weight of the total fatty acid content of the triglycerides. In one embodiment, the medium chain triglycerides may comprise up to 2% w/w C14 fatty acids.

The MCT solvent may comprise one, two, three, four or more different medium chain triglycerides. In one embodiment, the solvent comprises a medium chain triglyceride comprising esters of two C8 fatty acids and one C10 fatty acid. In one embodiment, the solvent comprises a medium chain triglyceride comprising esters of one C8 fatty acid and two C10 fatty acids. In one embodiment, the solvent comprises two different medium chain triglycerides, where a first medium chain triglyceride comprises esters of two C8 fatty acids and one C10 fatty acid and a second medium chain triglyceride comprises esters of one C8 fatty acid and two C10 fatty acids. In one embodiment, the solvent comprises a medium chain triglyceride which comprises 0 to 2% w/w C6 fatty acid, 50 to 65% w/w C8 fatty acid, 30 to 45% w/w C10 fatty acid, 0 to 2% w/w C12 fatty acid, based on the total fatty acid content of the medium chain triglyceride.

The triglycerides may be prepared by methods known in the art and are commercially available as MIGLYOL® 810, 812, 818, 829 (Sasol Germany GmbH, Witten, Germany) or NEOBEE® 1053, 895, M-5 (Stepan Company, Northfield, IL).

In another embodiment, the solvent is a propylene glycol diester of saturated vegetable fatty acids with chain lengths of C8 and C10 (caprylic and capric acid). An example of one such commercially available solvent is MIGLYOL® 840 (Sasol Germany GmbH, Witten, Germany).

Preferably, the composition comprises MCTs containing a mixture of C8 and C10 triglycerides in a ratio (C8:C10) of from about 55:45 to about 65:35, such as those available from Vigon International, Inc. (see ANNEX A).

Antioxidants

The present compositions employ at least one antioxidant, other than BCP. The at least one antioxidant can be vitamin E, which, for the sake of convenience, is used herein interchangeably with all-rac-α-tocopherol and α-tocopherol.

Vitamin E can refer to a group of eight water-insoluble compounds that include tocopherols and tocotrienols. The vitamin E used in the present compositions is the compound shown below, having the chemical formula C₂₉H₅₀O₂ (CAS #10191-41-0):

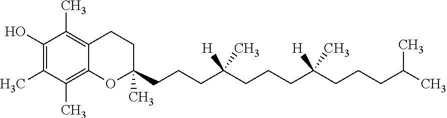 [SMILES: Cc1c(C)c2c(c(C)c1O)CC[C@@](C)(CCC[C@H](C)CCC[C@H](C)CCCC(C)C)O2]

Such compound is available from Spectrum Chemical Manufacturing Corporation of New Brunswick, New Jersey, U.S.A. (“Spectrum Chemical”). As with all commercial sources of chemical compounds, impurities may be present. Impurities include residual solvents of manufacture, e.g. toluene present in an amount less than 890 ppm. The Spectrum Chemical vitamin E was used in the below examples and has a purity of at least 99 wt. %.

Other antioxidants that can be used include polyphenols (phenolic acids, flavonoids, anthocyanins, lignans and stilbenes), carotenoids (xanthophylls and carotenes), propyl gallate, lecithin, curcumin, sesamin, sesamol, sesamolin, ascorbyl palmitate, butylated hydroxyanisole (BHA), and butylated hydroxytoluene (BHT), and monothioglycerol tert-butylhydroquinone (TBHQ). The antioxidant, e.g. vitamin E, can be used in an amount of from about 0.001, 0.01, 0.1, 0.2, 0.3, 0.4, 0.5, 0.6, or 0.7% w/v and up to about 10, 5, 4.5, 4.0, 3.5, 3.0, 2.5, 2.0, 1.5, 1.25, or 1% w/v.

The preparation may also contain an effective amount of antioxidant synergists, as are known in the art.

Additional Pharmaceutically Acceptable Excipients

The cannabinoid composition can comprise at least one additional pharmaceutically acceptable excipient. Examples of pharmaceutically acceptable excipients include and are not limited to viscosity modifiers, colouring agents, flavouring agents, sweeteners, taste masking agents, additional stabilizers or preservatives, absorption enhancers, and odorants. The choice and amount of the excipient(s) can be readily determined by one skilled in the art and will depend on the other ingredients in the composition, the desired properties of the final composition, oral route of administration, and need for a stable final product with the desired shelf-life.

The term “preservative” when used herein means those compounds that possess bactericidal and/or fungicidal properties.

Sweeteners that can be used in the present compositions are those that are lipid soluble. Examples include saccharin, alkoxy aromatic, oximes, sulfamic acids, dihydrochalcones, aspartyl malonates, succanilic acids, and mixtures thereof. The sweetener(s) can be present in an amount from about 0.001% w/v to about 5% w/v, about 0.01% w/v to about 1% w/v, or from about 0.25% w/v to about 0.5% w/v.

Flavouring agents that can be used are lipid soluble natural or synthetic compounds. These compounds can be present in an amount from about 0.1% w/v to about 5% w/v, about 0.1% w/v to about 1% w/v or from about 0.25% w/v to about 0.75% w/v. Examples include, without limitation, oil of sweet birch, oil of spearmint, oil of wintergreen, anise oil, dill oil, celery seed oil, citrus oils (e.g. lemon, orange, lime, tangerine and grapefruit oils), clove oil, peppermint oil, cassia, carrot seed oil, cola concentrate, ginger oil, angelica oil, vanillin, and combinations thereof.

Colouring agents that can be used in the present compositions include, without limitation, red, black and yellow iron oxides and FD&C dyes such as FD&C Blue No. 2, FD&C Red No. 40, and the like.

It is recognized that the ingredients or compounds used in the present composition may perform more than one function. For example, BCP functions as a solvent, antioxidant, flavouring agent, and pharmaceutically active agent. Therefore, the inclusion of any excipient, compound or ingredient into any one or more categories set forth above is not meant to limit the function of that excipient, compound or ingredient to the specified category.

Dosage Forms

The present compositions can take a variety of forms for oral administration to the gastrointestinal tract, including liquids, syrups, elixirs, soft gel capsules, and liquid-filled two-piece capsules. The person skilled in the art reading the present disclosure will appreciate what other forms the embodiments of the present compositions can take based on the present teachings.

As used herein, a soft gel capsule is an oral dosage form comprising a soft gel shell that can be made from gelatin or gelatin alternative, water, opacifier, and a plasticizer to lend flexibility, such as glycerin and/or sorbitol. Contained within the soft gel shell is a composition according to the present invention that provides a single or fractional dose of CBD. As used herein, a fractional dose refers to an amount that is less than a full dose so that, when provided as a capsule, a plurality of capsules will be required to provide a single dose. Typically, a fractional dose is at least 20%, 25%, 50% of a full dose.

Routes of Administration

The present compositions are administered to the gastrointestinal tract. Reference to “oral administration” is intended to cover all routes of administration to the gastrointestinal tract, including nasogastric administration.

The invention may be belier understood with reference to the examples below.

### EXAMPLES

The following ingredients were used to prepare solutions according to the present invention.

TABLE A

| Ingredient | Function in formulation | Supplier / Catalog |
|---|---|---|
| | | Number |
| (-)-cannabidiol (purified | Active Pharmaceutical | Dalton Pharma Services; |
| botanical) | Ingredient (API) | CAS #13956-29-1 |
| (-)-cannabidiol (synthetic) | Active Pharmaceutical | Noramco (now Purysis, |
| | Ingredient (API) | Inc.); 75407; CAS #13956-29-1 |
| (-)-cannabidiol (synthetic) | Active Pharmaceutical | Bio Vectra Inc.; Catalog |
| | Ingredient (API) | Number 7082; Lot Number |
| | | 49395; CAS #13956-29-1 |
| β-caryophyllene (BCP) | Co-solvent and antioxidant | Sigma Aldrich; W225207; |
| ≥80%, FC | | CAS # 87-44-5 |
| Coconut Oil | Principal Solvent | Spectrum Chem.: C0110; |
| | | CAS # 8001-31-8 |
| MCT (C8, C10 | Principal Solvent | Vigon International, Inc. of |
| triglycerides) | | Stroudsberg, Pennsylvania |
| | | under product code 507177 |
| Vitamin E (α-tocopherol, | Antioxidant | Spectrum Chemicals; CAS |
| FCC) ≥95.5% | | # 10191-41-0 |

ANNEX A contains the product specification sheets and/or certificates of analysis for the BCP and MCT in Table A. The CBD from Noramco (now Purisys, Inc.) and BioVectra Inc. are synthesized chemically, and present in a crystalline powdered form. The CBD from Dalton Pharma Services is ultra-purified botanically sourced CBD, also in a crystalline form. All forms of CBD shown in Table A are at least 98.5 wt. % pure. The following method of making an embodiment of the present composition can be used with any of the CBD products listed in Table A.

### Example 1

Coconut oil was warmed to 26° C. to 27° C. to liquefy the oil. A measured amount of the liquefied coconut oil was added to a main formulation vessel at room temperature. The solvent was stirred using a moderate vortex. A measured amount of BCP was added and the resultant mixture was stirred for 5 to 15 minutes until a homogenous mixture of co-solvents was formed. 15% of this co-solvent mixture was removed for use in rinsing containers of CBD and Vitamin E in a later step. The vortex speed in the main formulation vessel was increased and then a measured amount of crystalline CBD (in powder form) was added gradually from a vessel containing same. The vessel containing the CBD was then rinsed three times with part of the earlier retained co-solvent mixture and the rinse was added to the main formulation vessel. The main mixture was stirred for a further 30 minutes to four hours at a higher speed until the CBD dissolved completely. During stirring of the CBD mixture, the stirrer was stopped periodically in order to check for undissolved CBD particles. After formation of a clear homogeneous CBD solution, the antioxidant (Vitamin E as a thick oily liquid) was added to the main formulation vessel. The balance of the earlier retained co-solvent mixture was then used to rinse the antioxidant vessel. This last rinse was dispensed into the main vessel. The mixture was continued to be stirred at a lower speed for at least another 10 minutes until a clear and homogeneous final solution (Solution 1) was formed.

Solution 1

Solution 1 is summarized in Table 1 below.

TABLE 1

| INGREDIENT | AMOUNT (g) | AMOUNT (% w/v) |
|---|---|---|
| CBD | 27 | 22.67 |
| BCP | 29.9 | 25.11 |
| Coconut oil | 61.2 | 51.41 |
| Vitamin E | 0.95 | 0.81 |

The weight ratio of CBD:BCP was between 1:1 and 1:2 and the ratio of BCP:Coconut oil was between 1:2 and 1:3, or roughly 1:2.

Various modifications to the above method can be made. For example, the amounts of the ingredients used can be varied to make compositions according to the invention. The amount of vitamin E, for example, can be increased to about 1, 2, 3, 4 or 5% w/w and the amount of the solvent mixture (BCP and coconut oil) can be reduced by a corresponding amount.

### Example 2

Example 1 was repeated except that MCT (at room temperature) was used in place of coconut oil to produce Solution 2 summarized in Table 2 below.

TABLE 2

| INGREDIENT | AMOUNT (g) | AMOUNT (% w/w) |
|---|---|---|
| CBD | 27 | 22.67 |
| BCP | 29.9 | 25.11 |
| MCT | 61.2 | 51.41 |
| Vitamin E | 0.95 | 0.81 |

MCT is a liquid at room temperature. Unlike coconut oil, it does not need to be warmed, prior to use in the present method.

### Example 3

Three 500-ml batches of cannabidiol solutions (Solutions 3-5) were prepared having target concentrations of CBD of 30 mg/ml, 100 mg/ml and 250 mg/ml, respectively. In each solution, 1 part BCP, 2 parts coconut oil and 1% v/v of Vitamin E were employed.

The materials, target concentration, and composition of the CBD formulation prototype batches, 500 ml, are summarized in Table 3 below:

TABLE 3

| | Solution 3 (456.04 g | | | Solution 4 (459.78 g | | | Solution 5 (465.82 g | | |
|---|---|---|---|---|---|---|---|---|---|
| | total weight) | | | total weight) | | | total weight) | | |
| | | | % | | | % | | | % |
| Ingredient | mL | g | w/w | mL | g | w/w | mL | g | w/w |
| (−)-cannabidiol | n/a | 15.015 | 3.3 | n/a | 50.055 | 10.9 | n/a | 125.128 | 26.9 |
| BCP | 158 | 143.25 | 31.4 | 147 | 133.28 | 29.0 | 122 | 110.61 | 23.7 |
| Coconut Oil | 316 | 293.02 | 64.3 | 293 | 271.69 | 59.1 | 243 | 225.33 | 48.4 |
| Vitamin E | 5 | 4.75 | 1.0 | 5 | 4.75 | 1.0 | 5 | 4.75 | 1.0 |

| | | | |
|---|---|---|---|
| Target conc. | 30 | 100 | 250 |
| CBD (mg/mL) | | | |
| Approximate | 1:2 | 1:2 | 1:2 |
| Volume Ratio of | | | |
| BCP:Coconut oil | | | |

The concentration of CBD in Solutions 3-5 were assayed using a chromatographic technique and the results, shown in Table 4, are deemed to be acceptable for commercial pharmaceutical purposes.

TABLE 4

| | Target CBD | Measured CBD | |
|---|---|---|---|
| | concentration (mg/mL) | concentration | |
| Solution | [″Label Claim″] | (mg/mL) | % Label Claim |
| 3 | 30.0 | 28.8 | 96.0 |
| 4 | 100.0 | 100.3 | 100.3 |
| 5 | 250.0 | 249.7 | 99.9 |

### Example 4

Tests were performed to evaluation the stability of compositions according to the invention.

Solutions 6-8 (Lots 180-2842, 181-2842, and 182-2842) according to further embodiments of the invention were prepared and summarized in Table 5 below.

TABLE 5

| | Solution 6 | | Solution 7 | | Solution 8 | |
|---|---|---|---|---|---|---|
| | | % | | % | | % |
| Ingredient | g | w/w | g | w/w | g | w/w |
| CBD | 125.302 | 2.754 | 501.0 | 10.955 | 125.3 | 2.754 |
| BCP | 1459.2 | 32.070 | 1341.7 | 29.338 | 1459.3 | 32.072 |
| MCT | 2918 | 64.132 | 2683 | 58.668 | 2918 | 64.13 |
| Vit. E | 47.502 | 1.044 | 47.504 | 1.039 | 47.50 | 1.044 |

| | | | |
|---|---|---|---|
| Batch size | 5 | 5 | 5 |
| (L) | | | |
| CBD | 25 | 100 | 25 |
| target | | | |
| concen- | | | |
| tration | | | |
| (mg/mL) | | | |

Solutions 6 and 7 used synthetic CBD from Noramco (now Purisys, Inc.) (Lot #004803), while Solution 8 used synthetic CBD from BioVectra Inc. of Charlottetown, P.E.I. (Catalogue Number 7082, Lot Number 49395; CAS Registry Number 13956-29-1). Both the synthetic CBD from Noramco (now Purisys, Inc.) and from BioVectra Inc. were at least 98.5 wt. 00 pure.

Solutions 6-8 were each divided into 40 samples. 10 samples of each formulation were stored in 1 oz. amber glass boston round 200/400 bottles using as the closure a CR cap (20 mm, PE foam liner 20/40). These samples were stored right side up (RSU). Another 10 samples of each formulation were stored in the same containers oriented upside down (USD). Still another 10 samples of each formulation were stored in polyethylene (PET) boston round 20/400 bottles using as the closure a CR cap (20 mm Pictorial White, PE foam liner 20/400), oriented right side up (RSU). Finally, another 10 samples of each formulation were stored in the same bottles, oriented upside down (USD). All samples were stored at 40° C.±2° C. at relative humidity (RH) 75%+5% in a chamber (DCL 004065) according to protocol number STA-2842-7119.

The amount of CBD, BCP and THC was assayed using high performance liquid chromatography (HPLC) at time=0, 2, 4, 6, and 8 weeks. The percentage of the label claim (% LC) for the CBD and BCP was determined and the average results for all 10 bottles are summarized in Tables 6, 7, and 8. These tables also show the limit of the THC in the samples expressed in terms of ppm based on the total composition.

TABLE 6
Solution 6

| | | Time | | | | |
|---|---|---|---|---|---|---|
| | | T = | T = | T = | T = | T = |
| | | 0 wk | 2 wk | 4 wk | 6 wk | 8 wk |
| Glass Bot | CBD | 99.6 | 100 | 99.5 | 99.4 | 104.1 |
| RSU | BCP | 98.9 | 98.6 | 98.4 | 99.0 | 99.6 |
| | Limit THC (ppm) | N/A¹ | 0.14 | 0.003 | 0.06 | 0.07 |
| Glass Bot | CBD | 99.6 | 100.3 | 99.9 | 98.1 | 103.7 |
| USD | BCP | 98.9 | 98.8 | 98.6 | 97.8 | 99.2 |
| | Limit THC (ppm) | N/A¹ | 0.27 | 0.004 | 0.05 | 0.07 |
| PET Bot | CBD | 99.6 | 99.3 | 99.9 | 99.9 | 101.8 |
| RSU | BCP | 98.9 | 97.8 | 98.5 | 99.3 | 97.8 |
| | Limit THC (ppm) | N/A¹ | 0.15 | 0.004 | 0.05 | 0.10 |
| PET Bot | CBD | 99.6 | 100.2 | 100.5 | 94.8 | 102.3 |
| USD | BCP | 98.9 | 98.7 | 98 | 93.8 | 96.6 |
| | Limit THC (ppm) | N/A¹ | 0.13 | 0.007 | 0.05 | 0.10 |

¹The THC peak was misidentified due to insufficient chromatographic separation during the T = 0 testing.

The mean percentage LC for CBD for Solution 6 was 100.105±0.390224 std. deviation (across time periods), and ±1.597686 std. deviation (across bottle types and orientations).

TABLE 7
Solution 7

| | | Time | | | | |
|---|---|---|---|---|---|---|
| | | T = | T = | T = | T = | T = |
| | | 0 wk | 2 wk | 4 wk | 6 wk | 8 wk |
| Glass Bot | CBD | 99.1 | 99.2 | 92.1 | 97.6 | 101.5 |
| RSU | BCP | 99.4 | 98.9 | 92.0 | 98.5 | 98.2 |
| | Limit THC (ppm) | N/A¹ | 0.46 | 0.038 | 0.16 | 0.18 |
| Glass Bot | CBD | 99.1 | 97.1 | 99 | 98.2 | 101.2 |
| USD | BCP | 99.4 | 96.6 | 98.8 | 99 | 97.9 |
| | Limit THC (ppm) | N/A¹ | 0.41 | 0.025 | 0.15 | 0.16 |
| PET Bot | CBD | 99.1 | 99.3 | 99.1 | 95.8 | 102.7 |
| RSU | BCP | 99.4 | 99.1 | 99 | 96.7 | 99.5 |
| | Limit THC (ppm) | N/A¹ | 0.37 | 0.024 | 0.14 | 0.14 |
| PET Bot | CBD | 99.1 | 99 | 98.9 | 99.4 | 102.8 |
| USD | BCP | 99.4 | 98.6 | 98.7 | 100.3 | 99.5 |
| | Limit THC (ppm) | N/A¹ | 0.29 | 0.053 | 0.13 | 0.14 |

¹The THC peak was misidentified due to insufficient chromatographic separation during the T = 0 testing.

For Solution 7, the mean percentage LC for CBD was 98.965±0.699482 std. deviation (across time periods), and +1.671347 std. deviation (across bottle types and orientations).

TABLE 8
Solution 8

| | | Time | | | | |
|---|---|---|---|---|---|---|
| | | T = | T = | T = | T = | T = |
| | | 0 wk | 2 wk | 4 wk | 6 wk | 8 wk |
| Glass | CBD | 95 | 100.1 | 99.8 | 100.6 | 104.3 |
| Bot | BCP | 103.4 | 98.6 | 98.4 | 100.4 | 99.8 |
| RSU | Limit THC (ppm) | N/A¹ | 0.13 | 0.003 | 0.07 | 0.07 |
| Glass | CBD | 95 | 100.3 | 99.8 | 102.6 | 104.3 |
| Bot | BCP | 103.4 | 98 | 98.4 | 101.2 | 98.8 |
| USD | Limit THC (ppm) | N/A¹ | 0.07 | 0.002 | 0.06 | 0.08 |
| PET | CBD | 95 | 100.5 | 99.8 | 102.2 | 104.0 |
| Bot | BCP | 103.4 | 98.8 | 98.4 | 102.3 | 99.4 |
| RSU | Limit THC (ppm) | N/A¹ | 0.25 | 0.005 | 0.06 | 0.07 |
| PET | CBD | 95 | 100.2 | 100.6 | 102.4 | 104.6 |
| Bot | BCP | 103.4 | 97.9 | 97.8 | 101.6 | 99.2 |
| USD | Limit THC (ppm) | N/A¹ | 0.15 | 0.034 | 0.06 | 0.07 |

¹The THC peak was misidentified due to insufficient chromatographic separation during the T = 0 testing.

For Solution 8, the mean percentage LC for CBD was 100.305±0.219716 std. deviation (across time periods), and +3.062777 std. deviation (across bottle types and orientations).

The above results showed no trend of CBD loss over time, regardless of the type of vessel and cap closure used to store the CBD. Also, THC levels for all samples did not exceed 1 ppm during the period of the test. These results suggest that Solutions 6-8 should remain stable under long term storage conditions (at 25° C.±2° C. and relative humidity of 60° C. away from light) for at least 16 weeks and much longer if stored at colder temperatures, e.g. in a refrigerator (5° C.±3° C.) or in a freezer at −15° C.±10° C. The extremely low level of THC in the samples shows that these formulations should be safe and should provide consistent dosing in medical applications.

Solutions 6-8 were also inspected visually at time=0, 2, 4, 6, and 8 weeks. At all time points, all samples of these solutions appeared as a homogeneous, pale, yellow clear liquid and passed the visual inspection tests.

Example 4 employed synthetic CBD. However, the same stability results can be expected when substituting the synthetic CBD with CBD purified by Dalton Pharma Services of Toronto, Ontario, Canada. Such product is (−)-cannabidiol (CAS #13956-29-1) (the Dalton Pharma CBD) described above. The Dalton Pharma CBD is equivalent to the synthetic CBD used in the above experiments for the purposes of the present specification.

Methods of Treating a Condition, Disorder, Disease or Symptom Thereof

The present compositions are intended to be used in any therapy in which cannabidiol is indicated, including, without limitation, the treatment and/or prevention of cancer (e.g. Glioblastoma multiforme), cardiovascular disease (e.g. acute myocarditis, heart failure, including heart failure with preserved ejection fraction (HFpEF)), anxiety, autism, seizures, chronic pain, psychosis, arthritis, and other diseases or disorders involving inflammation.

The amount of CBD to be administered will vary by body weight and nature of the condition. The dosage regime can entail the administration of 0.1 to 30 mg of CBD/kg body weight per day, preferably 10-20 mg of CBD per kg body weight per day. The dose can be divided for administration 2, 3, or 4 times a day. The CBD composition can be administered every 2, 3, 4, 5, 6, or 7 days, for a period of from 1, 2, 3, 4, 5, 6, 7, 8, 9, or 10 weeks. Lower doses of CBD can be administered for longer periods of time to promote health, rather than to treat a disease condition.

The foregoing description of embodiments is by way of example only and is not intended to limit the scope of the invention as herein described and claimed.

1. An oral cannabidiol composition comprising:
a. cannabidiol (CBD) in a concentration of from about 1 w/v % to about 35 w/v %;
b. a lipophilic carrier in a concentration of from about 60 w/v % to about 98.9 w/v 00 wherein the lipophilic carrier comprises β-caryophyllene (BCP) and at least one additional lipophilic solvent, wherein the volume ratio of BCP to the at least one additional lipophilic solvent is from about 1:1 to about 1:3;
c. at least one additional antioxidant in a concentration of from about 0.1% w/v to about 5% w/v; and
d. from 0% w/v to an effective amount of at least one pharmaceutically acceptable excipient;
wherein the CBD is a purified botanical isolate that is at least 98 wt. % pure, and wherein the composition is substantially free of THC.

2. (canceled)

3. (canceled)

4. The composition of claim 1, wherein the CBD is at least 98.5 wt. % pure.

5. The composition of claim 4, wherein the CBD is at least 99 wt. % pure.

6. The composition of claim 5, wherein the CBD is at least 99.5 wt. % pure.

7. The composition of claim 1, wherein THC is not present, or is present in an amount not exceeding 10 ppm, based on the total composition.

8. The composition of claim 1, wherein the at least one additional lipophilic solvent is selected from the group consisting of medium chain (C6-C12) triglycerides (MCT), coconut oil, sesame oil, fish oil, avocado oil, oils from nuts and seeds, corn oil, peanut oil, safflower oil, soybean oil, and palm kernel oil.

9. The composition of claim 1, wherein the at least one additional antioxidant is selected from the group consisting of α-tocopherol (vitamin E), polyphenols, carotenoids, propyl gallate, lecithin, curcumin, sesamin, sesamol, sesamolin, ascorbyl palmitate, butylated hydroxyanisole (BHA), butylated hydroxytoluene (BHT), and monothioglycerol tert-butylhydroquinone (TBHQ).

10. The composition of claim 1, wherein the volume ratio of BCP to the at least one additional lipophilic solvent is about 1:2.

11. The composition of claim 1, wherein the CBD is present in an amount from about 2% w/v to about 25% w/v.

12. The composition of claim 11, wherein the CBD is present in an amount from about 9% w/v to about 23% w/v.

13. The composition of claim 1, wherein the at least one antioxidant consists of α-tocopherol (vitamin E).

14. The composition of claim 13, wherein the α-tocopherol is present in an amount from about 0.5% w/v to about 1.5% w/v.

15. The composition of claim 1, wherein the at least one additional lipophilic solvent consists of a mixture of C8 and C10 triglycerides.

16. The composition of claim 15, wherein the weight ratio of the C8 triglyceride to the C10 triglyceride is from about 55:45 to about 65:35.

17. A stable oral cannabidiol composition consisting of:
a. botanically sourced CBD having a purity of at least 98.5 wt. %, in an amount from about 2% w/v to about 12% w/v;
b. BCP in an amount from about 28% w/v to about 30% w/v;
c. a mixture of C8 and C10 triglycerides in an amount from about 56% w/v to about 60% w/v;
d. α-tocopherol (vitamin E) in an amount from about 0.5% w/v to about 1.5% w/v; and
e. from 0% w/v to an effective amount of at least one pharmaceutically acceptable excipient;
wherein THC is absent or present as an impurity in an amount less than 10 ppm based on the total composition.

18. The composition of claim 17, wherein the weight ratio of the C8 triglyceride to the C10 triglyceride is from about 55:45 to about 65:35.

19. The composition of claim 1, wherein the composition is substantially free of other cannabinoids, other terpenes, other solvents, essential oils, surfactants, emulsifiers, alcohols, and other pharmaceutically active ingredients.

20. A method of stabilizing botanically sourced CBD in an oral composition containing same, the method comprising dissolving botanically sourced CBD having a purity of at least 98 wt. % in a lipophilic carrier consisting of BCP and at least one additional lipophilic solvent, adding and mixing in an antioxidant other than BCP, wherein,
a. the volume ratio of the BCP to the at least one additional lipophilic solvent is from about 1:1 to about 1:3; and
b. said antioxidant other than BCP is present in an amount from about 0.5% w/v to about 1.5% w/v.

21. The method of claim 20, wherein the volume ratio of BCP to the at least one additional lipophilic solvent is about 1:2.

22. The method of claim 20, wherein the at least additional lipophilic solvent is MCT comprising C8 and C10 triglycerides, and wherein the weight ratio of the C8 triglyceride to the C10 triglyceride is from about 55:45 to about 65:35.
